(19) United States
(12) Patent Application Publication
(10) Pub. No.: US 2006/0004298 A1
(43) Pub. Date: Jan. 5, 2006

(54) Software controlled electromyogram control systerm

(76) Inventors: Philip R. Kennedy, Duluth, GA (US); Dinal Andreasen, Marietta, GA (US); Yian Chuin Cheng, Alpharetta, GA (US); Richard Montricul, Atlanta, GA (US); Kristan R. Wagner, Atlanta, GA (US); Ronnie J. H. Wilmink, Marietta, GA (US); Edward Joseph Wright, Atlanta, GA (US)

Correspondence Address: BRYAN W. BOCKHOP, ESQ., 2375 MOSSY BRANCH DR., SNELLVILLE, GA, 30078, US

(21) Appl. No.: 10/881,923

(22) Filed: Jun. 30, 2004

(51) Int. Cl. A63F 13/00; A61B 5/04; G09B 21/00

(52) U.S. Cl. 600/546; 463/36; 340/825.19

(57) ABSTRACT

A system for enabling a user to exert control with bioelectrical impulses via an input from the user includes a first electromyogram interface, a computer display and a computer. The first electromyogram interface to the user is in communication with a first source of bioelectrical impulses from the user. The computer display is capable of displaying a cursor. The computer is in communication with the electromyogram interface and the computer display, and is programmed to sense a first input from the first electromyogram interface, change a first computer control attribute in response to a state change sensed in the first input, and generate a preselected cursor action in response to a change in the first computer control attribute.

### BACKGROUND

1. Field of the Invention

The present invention generally relates to electromyogram systems and, more specifically, to an electromyogram interface.

2. Description of the Related Art

Muscle paralysis affects over one hundred thousand people in the United States and approximately one million people worldwide. One approach used to provide assistance to paralyzed people has been described by the U.S. Pat. No. 4,852,573, which is hereby incorporated by reference.

One class of patients who face severe difficulties in their daily lives is those with locked-in syndrome. Locked-in syndrome patients generally have a cognitively intact brain and a completely paralyzed body. They are alert but cannot move or talk. They face a life-long challenge to communicate. Some patients may use eye movements, blinks or remnants of muscle movements to indicate binary signals, such as “yes” or “no.” To enhance communication with these patients, several devices have been developed including electroencephalographic (EEG) and electromyographic (EMG) control of a computer. These systems can provide patients with the ability to spell words.

Typical EMG control devices receive bioelectrical impulses from EMG sensors attached to the user's body. The EMG sensors sense small electrical impulses generated by motor nerves in various parts of the user's body, such as the forearms and the jaw.

Current typical EMG control systems use a single input to control scanning movement of a cursor over an image of a keyboard that is displayed on a computer screen. The cursor scans across the rows of the keyboard image and the user asserts an EMG impulse when the cursor is over a desired location on the keyboard. However, such systems do not provide movement control of the cursor other than keyboard scanning.

Thus, there is a need for a system and method that enable multipurpose control of a cursor using EMG inputs.

### SUMMARY OF THE INVENTION

The invention, in one aspect, includes a system for enabling a user to exert control with bioelectrical impulses via an input from the user. The system includes a first electromyogram interface, a computer display and a computer. The first electromyogram interface to the user is in communication with a first source of bioelectrical impulses from the user. The computer display is capable of displaying a cursor. The computer is in communication with the electromyogram interface and the computer display. The computer is programmed to sense a first input from the first electromyogram interface, change a first compuuter control attribute in response to a state change sensed in the first input, and generate a preselected action in response to a change in the first computer control attribute.

In another aspect, the invention includes a method of validating an electromyogram signal in which a counter is incremented at a first rate of a first preselected number of counts per second if the electromyogram signal has been asserted. The counter decremented at a second rate of a second preselected number of counts per second if the electromyogram signal has not been asserted and if the counter has a value not equal to zero. An electromyogram state change signal is asserted if the counter has a value of not less than a predetennined threshold value that is no equal to zero.

In another aspect, the invention includes a method of processing electromyogram information on a computer-based system that includes a computer display. A cursor displayed on the computer display is caused to move in response to a first assertion of an electromyogram signal. A sleep-mode icon is displayed on the display. The computer-based system enters into a sleep-mode state when the cursor is in a position corresponding to the sleep-mode icon. A predetermined set of functions controlled by the computer-based system are disabled upon entering the sleep-mode state. A second assertion of the electromyogram signal is sensed. The predetermined set of functions is re-enabled when the second assertion of the electromyogram signal indicates that a predetermined electromyogram state has been changed.

In another aspect, the invention includes a method of processing electromyogram information on a computer-based system that includes a computer display. A cursor displayed on the computer display is caused to move in response to a first assertion of an electromyogram signal. A special mode icon is displayed on the display. The computer-based system enters into a special mode state when the cursor is in a position corresponding to the special mode icon such that a predetermined electromyogram state change signal has been asserted. A special mode indication is generated when the computer-based system has entered the special mode state.

In another aspect, the invention includes a method of processing electromyogram information from a user in which a first electromyogram signal corresponding to a first condition from the user is measured. A second electromyogram signal corresponding to a second condition, which contrasts with the first condition, from the user is measured. A fast Fourier transform is applied to the first signal, thereby generating a first frequency domain signal. A fast Fourier transform is applied to the second signal, thereby generating a second frequency domain signal. The first frequency domain signal and the second frequency domain signal are compared according to predefined criteria, thereby creating a filter function. A fast Fourier transform is applied to a real-time electromyogram signal, thereby generating a real time frequency domain signal. The filter function is applied to the real-time frequency domain signal, thereby generating a real-time filtered signal. An inverse fast Fourier transform is applied to the real-time filtered signal, thereby generating a real-time filtered time domain signal corresponding to the real-time electromyogram signal.

In another aspect, the invention includes a device for interfacing an electromyogram to a computer. The device is operatively coupled to a power supply, a first electromyogram channel input, a first output that is capable of transmitting a signal from the first electromyogram channel input to the computer, a first computer signal input that is capable of receiving a data signal from the computer, a first switch output and a first relay. The first relay is activated by the first computer signal input and electrically couples the power supply to the first switch output when a first signal is asserted at the first computer signal input. The first signal indicates that a bioelectrical impulse has been sensed by the first electromyogram channel input.

In yet another aspect, the invention includes an electromyogram interface for sensing an input from a user. The interface includes contact with a bioelectrical impulse sensor that is capable of generating a first signal when a bioelectrical impulse is asserted and a piezoelectric member that is capable of generating a second signal when subjected to a mechanical force corresponding to a muscle movement. A detection system that is responsive to the bioelectrical impulse sensor and the piezoelectric member determines if the input from the user has been asserted based on the first signal and the second signal. The detection system is also capable of determining if either the bioelectrical impulse sensor or the piezoelectric member is malfunctioning and, thereby determining if the input from the user has been asserted even when one of the bioelectrical impulse sensor or the piezoelectric member is malfunctioning.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram of one embodiment of an electromyogram interface.

FIG. 2 is a side view of one embodiment of an electromyogram interface.

FIG. 3 is a front view of a computer display.

FIG. 4 is a flow chart showing a procedure used to verify assertion of an EMG signal.

FIG. 5 is a chart showing a progression of a counter used in verifying assertion of an EMG signal.

FIG. 6 is a view of a display with a wrap-around cursor.

FIG. 7 is a view of a display with a direction-selectable cursor.

FIG. 8 is a view of a display with a reversing cursor.

FIG. 9 is a view of a display with a rosette-type cursor.

FIG. 10 is a view of a display with a rotating cursor.

FIG. 11 is a view of a display with a three-mode cursor.

FIG. 12 is a schematic diagram of an electromyogram-computer interface circuit.

FIG. 13A is a block diagram of a filter generator.

FIG. 13B is a set of three histograms showing different frequency components of an electromyogram signal for an “ON” condition and an “OFF” condition, and the difference between the “ON” condition and the “OFF” condition.

FIG. 13C is a flow chart for a filter generation procedure.

FIG. 13D is a block diagram of a filtering mechanism.

FIG. 14A is a histogram of the frequency components of a “CALIBRATION ON” signal.

FIG. 14B is a histogram of the frequency components of a “CALIBRATION OFF” signal.

FIG. 14C is a histogram of the difference between the frequency components of “CALIBRATION ON” signal and the “CALIBRATION OFF” signal, and resulting filter values.

FIG. 14D is a histogram of the frequency components of a real time signal.

FIG. 14E is a histogram of the “CALIBRATION OFF” signal, as shown in FIG. 14B, shown again for clarity.

FIG. 14F is a histogram of the difference between the frequency components of real time signal and the “CALIBRATION OFF” signal and the results of the difference values being multiplied by filter values.

FIG. 14G is a histogram showing comparison of a sum of the multiplied values of FIG. 14F to an activation threshold.

### DETAILED DESCRIPTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

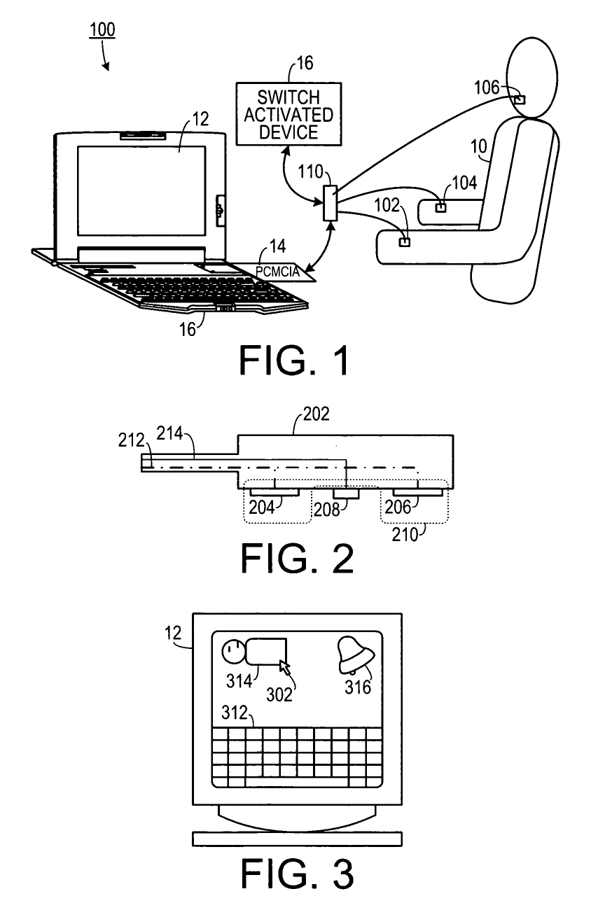

As show in FIG. 1, one embodiment of the invention includes a system 100 for enabling a user 10 to exert control with bioelectrical impulses. The system 100 allows the user 10 to control such devices as a computer 16, a computer display 12, and a switch-activated device 16 (such as a relay-controlled lamp or fan). The user 10 communicates with the system 100 via a plurality of bioelectrical impulse sensors 102, 104, 106, such as electromyogram (EMG) interfaces. Two of the bioelectrical impulse sensors 102 and 104 may be applied to respective limbs of the user 10, whereas a third bioelectrical impulse sensor 106 may be applied to another area of the user's 10 body, such as the neck or jaw. The computer 16 is programmed to sense one or more inputs from the electromyogram interfaces 102, 104, 106 and change a computer control attribute in response to a state change sensed in the inputs. The computer 16 causes a preselected action in response to a change in the computer control attribute. Several illustrative examples of computer control attributes include, but are not limited to: the movement of a cursor; assertion of default action key, such as space bar or letter key; and control of a device controlled by a computer, such as a lamp.

The bioelectrical impulse sensors 102, 104, 106 communicate with the computer 16 through an interfacing device 110, which communicates with the computer 16 via an interface card 14 (such as a PCMCIA card or a USB card). In one embodiment, a bloelectrical impulse sensor 202, as shown in FIG. 2, may include two electrical contacts 204, 206 that form a bioelectrical impulse sensor 210 (such as an EMG sensor) and a piezoelectric member 208. The piezoelectric member 208 is capable of generating a piezoelectric signal 214 when subjected to a mechanical force corresponding to a muscle movement. The bioelectrical impulse sensors 210 are capable of generating a bioelectric signal 212 when the user 10 generates a bioelectrical impulse, such as by attempting to flex a muscle. The computer 16 may be programmed to determine whether the user 10 has asserted an input based on the piezoelectric signal 214 and the bioelectric signal 212. The system is capable of determining if either the bioelectrical impulse sensor 210 or the piezoelectric member 208 is malfunctioning. The algorithm could be as simple as accepting the assertion of either bioelectrical impulse sensor 210 or the piezoelectric member 208 as an assertion of a signal (e.g., “OR'ing” the signals from the bioelectrical impulse sensor 210 and the piezoelectric member 208). The system could also employ an algorithm that considers recent past history to determine if a sensor is malfunctioning.

As shown in FIG. 3, the computer control attribute could include movement of a cursor 302 (in which the system is in a mouse emulator mode) or selection of keys of a keyboard image 312 on the display 12 (when the system is in a scanning input mode). Assertion of the bioelectric input may also correspond to a mouse “click” that causes a computer action in a manner similar to the clicking of a mouse button, which is also a computer control attribute.

The display 12 could display special mode state action icons, such as a sleep mode icon 314 and an alarm mode icon 316. The sleep mode icon 314 can be used to put the computer 16 into a sleep mode, wherein the computer disables a predetermined set of functions from the time it is invoked until the user 10 indicates that the sleep mode is to be terminated. Invoking the sleep mode may be done by positioning the cursor 302 over the sleep mode icon 314 using EMG control and asserting an EMG signal while the cursor 302 is positioned over the sleep mode icon 314. The sleep mode can be used to disable computer noises and other computer-controlled stimuli, such as telephone calls and lamps. Such stimuli might interfere with the user's sleep and, therefore, the user may use the sleep mode to reduce disturbances. The user can exit the sleep mode by reasserting the EMG signal while the cursor 302 is positioned over the sleep mode icon 314.

The alarm mode icon 316 can be used to put the computer 16 into an alarm mode, wherein the computer generates a signal (such as a loud noise or an indicator on an alarm panel) indicating that the user 10 seeks assistance. Similarly to the sleep mode, the alarm mode may be invoked when the user 10 positions the cursor 302 over the alarm mode icon 316 and asserts an EMG signal.

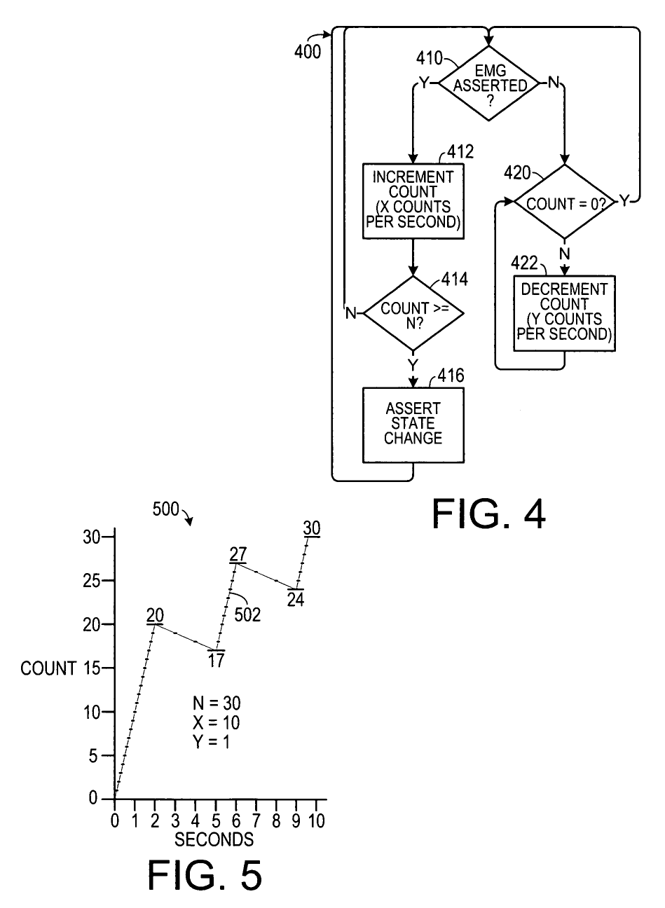

As shown in FIG. 4, one method 400 of verifying the assertion of the EMG signal and distinguishing it from spurious inputs, involves counting the amount of time that the EMG signal is asserted versus the amount of time that it is not asserted. This method 400 filters out signals of short duration, yet allows for short periods of rest due to fatigue. Initially, the system determines 410 if an EMG signal has been asserted. If not, the system determines 420 if the counter for the amount of time the signal has been asserted is equal to zero. If it is zero, then control passes back to step 410. Otherwise, the counter is decremented 422 by a predetermined amount per second until the counter equals zero. If, at step 410, an EMG signal is sensed, then the system increments 412 the counter by a predetennined amount per second and then determines 414 if the counter has reached a predetermined threshold. If not, then control passes back to step 410. Otherwise, the system has reached the threshold and, thus, asserts a state change 416, such as entering or exiting the sleep mode or the alann mode.

As shown in FIG. 5, a graph 500 showing a typical process in which the counter 502 (N) is incremented resulting in the assertion of a state change, the rate at which the counter is incremented (X) may be greater than the rate at which it is decremented (Y). This allows for user fatigue: the counter (N) goes up rapidly while the EMG signal is asserted, but goes down relatively slowly allowing for short periods of rest. The figures for X, Y and N may be adjusted according to the specific ability of a individual user.

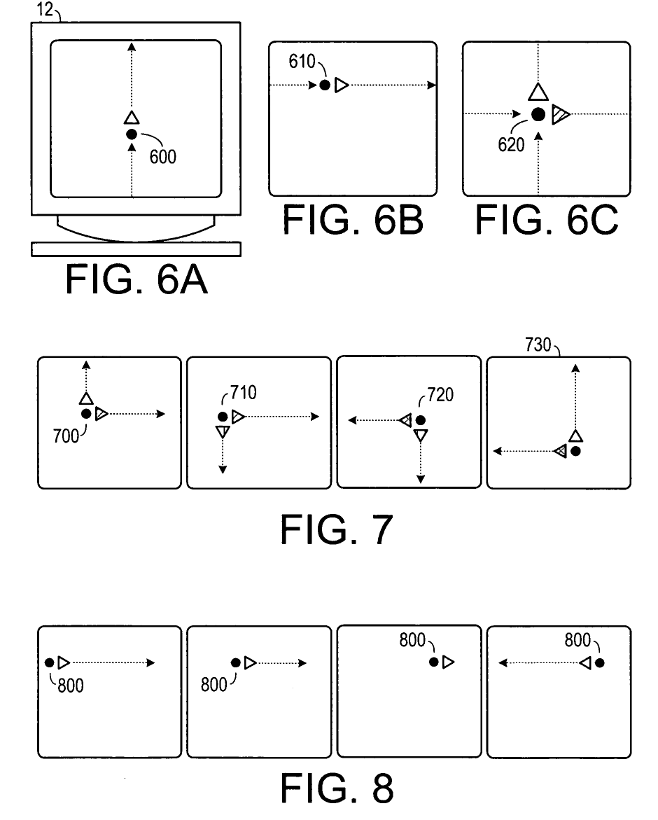

As shown in FIGS. 6-11, several different cursor movement methods are shown. In FIG. 6A, the cursor is a wrap-around type cursor 600 that starts moving upwardly when the user asserts an EMG signal and then stops when a second EMG signal is asserted. When the cursor reaches the top of the screen 12, it wraps around to begin upward movement from the bottom. As shown in FIG. 6B, a second EMG input may be used to switch from a vertical movement cursor 600 to a horizontal movement cursor 610. A combined cursor 620 is shown in FIG. 6C, in which a first EMG input controls movement of the cursor and a second EMG input controls the direction of movement. A rotating combined cursor 700 is shown in FIG. 7, in which the cursor may initially allow movement up and to the right. An assertion of a first EMG input could cause the cursor to become one that moves to the right and down 710, another assertion of the first EMG input could cause the cursor orientation to rotate 90° 720, and a subsequent assertion could cause another rotation 730. A second EMG input controls whether the cursor moves horizontally or vertically and a third EMG input starts and stops movement. As shown in FIG. 8, a back and forth moving cursor 800 uses a first EMG input to control left or right (or up or down) movement and a second EMG input to initiate and stop movement.

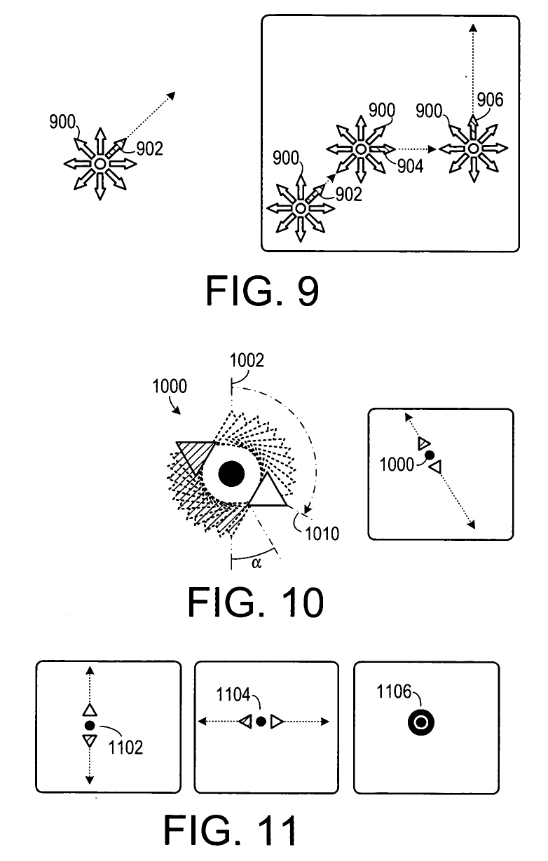

A rosette-type cursor 900 is shown in FIG. 9. This type of cursor includes a plurality of arrows radiating out of a central locus. One of the arrows 902 is highlighted at any given time and the highlighted arrow rotates about the locus either as a result of passage of time or assertion of an EMG input. Once the highlighted arrow is pointing in the desired direction, the user asserts an EMG signal to initiate movement. Once a desired waypoint is reached, the user may select a second direction 904 and, subsequently, a third direction 906. This selection process may continue until the desired location for the cursor 900 is reached.

A rotating cursor 1000 is shown in FIG. 10, in which the cursor 1000 rests along a first axis 1002 during inactive periods. When the user asserts an EMG signal, the cursor 1000 begins to rotate from the first axis. When the cursor 1000 reaches a desired orientation, the user releases the EMG signal (or the user may reassert it, depending on the configuration) and then asserts a second EMG signal to select between the two directions pointed to by the arrows. A subsequent assertion of an EMG signal causes movement of the cursor 1000. The cursor 1000 may be limited to rotate no further than angle α that is less than 180° from the first axis 1002 so as to prevent confusion by the user.

As shown in FIG. 11, the cursor may be a multi-mode cursor, with each assertion of a first EMG signal changing the cursor from a first mode 1102 to a second mode 1104, and then to a third mode 1106. The first mode 1102 facilitates vertical movement, the second mode 1104 facilitates horizontal movement and the third mode 1106 presents a target symbol that corresponds to initiating an activity, such as activating a process represented by an icon under the target symbol.

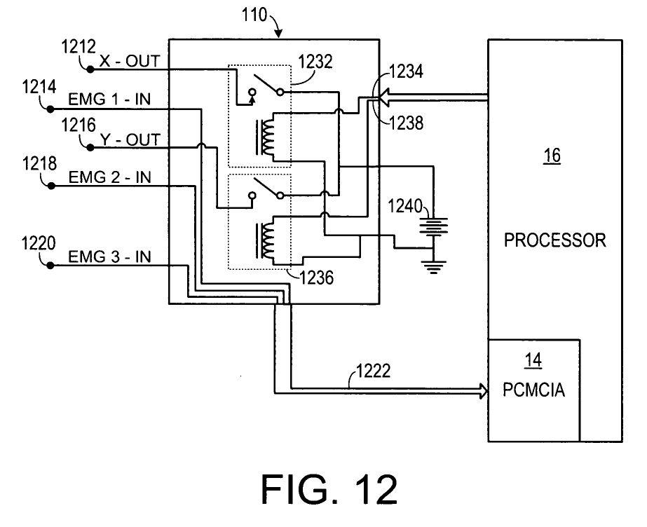

As shown in FIG. 12, one embodiment of the interfacing device 110 includes a first EMG input 1214, a second EMG input 1218 and a third EMG input 1220, which are all fed into a data bus 1222 in communication with the computer 16 via the PCMCIA card 14. This embodiment also includes an X output 1212 and a Y output 1216. The X output 1212 and the Y output 1216 may be used to control external devices or to send signals to ports other that the PCMCIA card 14 of the computer 16. A first relay 1232 is controlled by a first data line 1234 from the computer 16 and selectively couples the X output 1212 with a power supply 1240. Similarly, a second relay 1236 is controlled by a second data line 1238 from the computer 16 and selectively couples the Y output 1216 to the power source 1240.

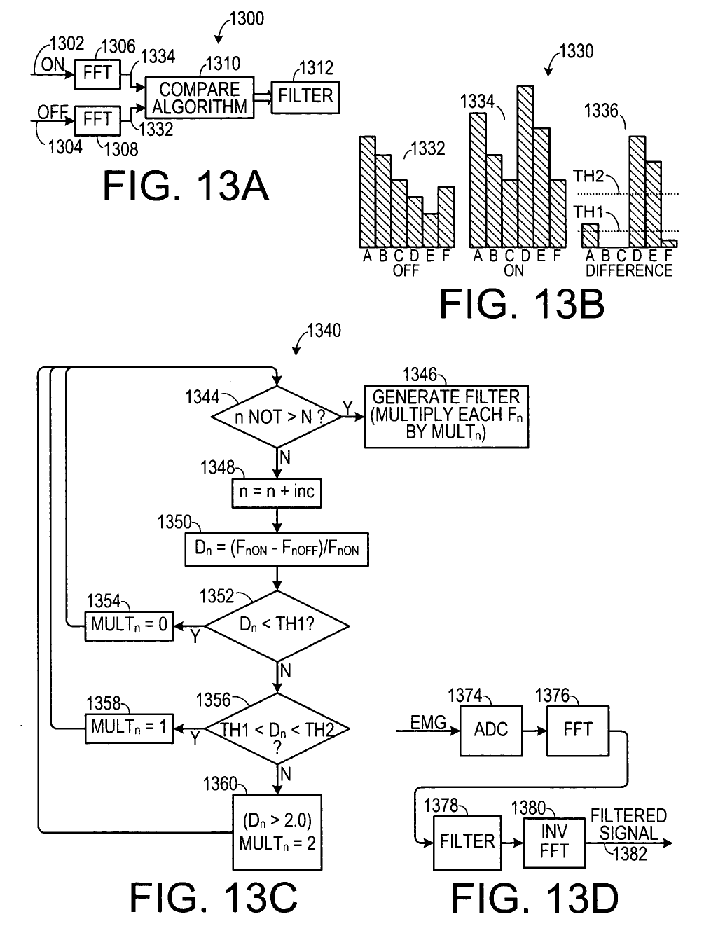

As shown in FIGS. 13A-13D, one embodiment of the system uses a filter generator 1300 to distinguish between states of electromyogram inputs from the user. In calibrating the system, at least one “ON” input 1302 corresponding to the user's intent that an electromyogram signal be asserted (or another conditional input from the user) is measured. Similarly, at least one “OFF” input 1304 corresponding to the user's intent that an electromyogram signal be unasserted (or another contrasting conditional input from the user) is also measured. The “ON” and “OFF” inputs are digitized using an analog-to-digital converter. A fast Fourier transform (FFT) 1306 is applied to the ON input 1302, thereby generating a first frequency domain signal 1334. (The frequency domain signals are represented in FIG. 13B as a plurality of frequency domain groupings A-F, in which each grouping corresponds to a range of frequencies and the value of the signal corresponds to an average intensity of each frequency range, which correspond to the frequency components of the underlying time domain signal.) An FFT 1308 is applied to the OFF input 1304, thereby generating a second frequency domain signal 1332. (Although FIG. 13A shows two FFT's, it is understood that the FFT function may be performed by a single FFT circuit at different times, without departing from the scope of the claims. It is also understood that any one of several commonly known FFT algorithms may be employed.) The first frequency domain signal 1334 and the second frequency domain signal 1332 are compared according to predefined criteria, thereby creating a filter function 1312.

As shown in FIG. 13B, in one embodiment, the comparison criteria used may include subtracting each frequency domain grouping A-F of the second frequency domain signal 1332 from the corresponding frequency domain grouping A-F of the first frequency domain signal 1334, which results in a plurality of difference values 1336. As shown in FIG. 13C, these difference values 1336 are used by a filter generation method 1340 to generate the filter. In one embodiment of the filter generation method 1340, a processor compares each difference value of the plurality of difference values 1336 to a first threshold TH1 and a second threshold TH2 to determine a multiplying factor. Initially, the system determines 1344 if each difference value has been evaluated. If not, the system increments 1348 a counter that points to the next difference value to be evaluated. The system normalizes 1350 the difference value as a ratio of the difference value divided by the ON value. Next, the system determines 1352 if the normalized difference value Dₙ is less than the first threshold TH1 (which could be 0.5, for example) and, if so, then assigns 1354 a multiplier factor Multₙ of “0” for the corresponding frequency range grouping. If the normalized difference value Dₙ is not less than the first threshold TH1, then the system determines 1356 if the normalized difference value Dₙ is between the first threshold TH1 and a second threshold TH2 (which could be 2.0, for example). If so, the system assigns 1358 a multiplier factor Multₙ of “1” for the corresponding frequency range grouping, otherwise the system assigns 1360 multiplier factor Multₙ of “2” for the corresponding frequency range grouping. Once each difference value has been evaluated, then the system generates the filter function 1346, which is essentially a table that links each multiplier factor Multₙ, to its corresponding frequency range grouping, n.

Employment of the filter 1378 is shown in FIG. 13D. The system receives impulses from an EMG input and converts the signal into a digital signal using an analog-to-digital converter 1374. The digital signal is converted into a frequency domain signal using a fast Fourier transform (FFT) 1376, the frequency domain components of the frequency domain signal are multiplied by corresponding the multiplier factors Multₙ by the filter 1378 and the resulting values are converted back to the time domain with an inverse FFT 1380, thereby generating a filtered digital signal 1382.

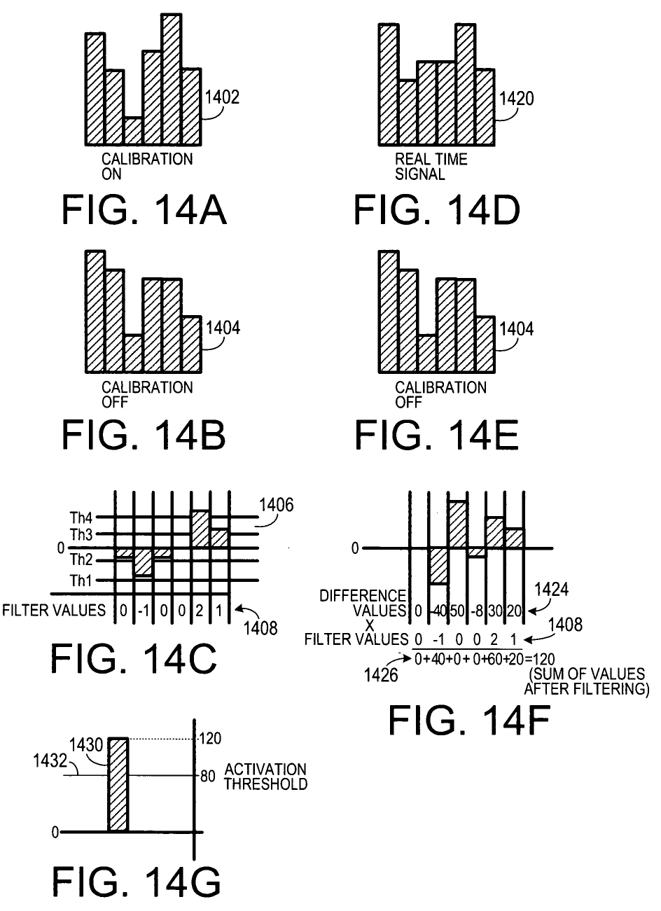

As shown in FIGS. 14A-14G, in another method of filtering the EMG input signal, during the calibration step, an “ON” calibration signal is measured and converted into an “ON” frequency domain calibration signal 1402 and an “OFF” calibration signal is measured and converted into an “OFF” frequency domain calibration signal 1404. The “OFF” frequency domain calibration signal 1404 is subtracted from the “ON” frequency domain calibration signal 1402 and the resulting value is compared to a plurality of thresholds (Th1, Th2, Th3, and Th4), which gives rise to the assignment of a corresponding plurality of filter values 1408 to each frequency component.

In filtering the real time EMG signal, the real time EMG is converted into a real time frequency domain signal 420 from which the “OFF” frequency domain calibration signal 1404 is subtracted. The resulting real time difference values 1422 are then multiplied by the filter values 1408 that were calculated during the calibration step. The resulting values 1426 are added to generate a sum value 1430. The sum value 1430 is then compared to an activation threshold 1432. If the sum value 1430 is greater than the activation threshold 1432 then the system accepts the EMG input as having been asserted, otherwise the system does not accept the EMG input as having been asserted.

While the invention has been particularly shown and described with reference to a embodiment shown herein, it will be understood by those skilled in the art that various changes in form and detail maybe made without departing from the spirit and scope of the present invention as set for the in the following claims. Furthermore, although elements of the invention may be described or claimed in the singular, the plural is contemplated unless limitation to the singular is explicitly stated. While the examples above use EMG signals as the bioelectrical input to the system, it is understood that other types of bioelectrical signals my be used without departing from the scope of the invention.

What is claimed is:

1. A system for enabling a user to exert control with bioelectrical impulses via an input from the user, comprising:
a first electromyogram interface to the user in communication with a first source of bioelectrical impulses from the user;
a. a computer display capable of displaying a cursor; and
b. a computer, in communication with the electromyogram interface and the computer display, programmed to execute the following steps:
i. sense a first input from the first electromyogram interface; change a first computer control attribute in response to a state change sensed in the first input; and
ii. generate a preselected action in response to a change in the first computer control attribute.

2. The system of claim 1, wherein the first computer control attribute comprises a cursor attribute.

3. The system of claim 1, wherein the first computer control attribute comprises a keyboard attribute.

4. The system of claim 1, wherein the first computer control attribute indicates a direction of cursor movement.

5. The system of claim 1, wherein the first computer control attribute indicates a selection between a mouse emulation input mode and a scanning input mode.

6. The system of claim 1, wherein the first computer control attribute indicates a selection between a cursor movement mode and a cursor click mode.

7. The system of claim 1, further comprising a second electromyogram interface to the user in communication with a second source of bioelectrical impulses from the user, different from the first source of bioelectrical impulses.

8. The system of claim 7, wherein the computer is programmed to execute the following steps:
a. sense a second input from the second electromyogram interface;
b. change a second computer control attribute in response to a state change sensed in the second input; and
c. generate a preselected cursor action in response to a change in the second computer control attribute.

9. The system of claim 8, wherein the first computer control attribute comprises a selection of cursor direction and wherein the second computer control attribute comprises a selection of cursor movement.

10. The system of claim 9, wherein cursor comprises an image of a first arrow and an image of a second arrow and wherein the selection of cursor direction is accomplished according to the following steps:
a. displaying the image of the first arrow pointing in a first direction on the computer display;
b. displaying the image of the second arrow pointing in a second direction, different from the first direction, on the computer display;
c. indicating a change selection of arrow from the first arrow to the second arrow or from the second arrow to the first arrow each time the first input from the first electromyogram interface is sensed; and
d. causing the cursor to move in the direction of a currently selected one of the first arrow and the second arrow when the second input from the second electromyogram interface is sensed.

11. The system of claim 10, wherein the direction of the first arrow is transverse to the direction of the second arrow.

12. The system of claim 7, further comprising a third electromyogram interface to the user in communication with a third source of bioelectrical impulses from the user, different from the first source of bioelectrical impulses and the second source of bioelectrical impulses.

13. The system of claim 12, wherein at least one of the first electromyogram interface, the second electromyogram interface or the third electromyogram interface comprises:
a. a bioelectrical impulse sensor, capable of generating a first signal when a bioelectrical impulse is asserted;
b. a piezoelectric member, capable of generating a second signal when subjected to a mechanical force corresponding to a muscle movement; and
c. a detection system, responsive to the bioelectrical impulse sensor and the piezoelectric member, that determines if the input from the user has been asserted based on the first signal and the second signal, the detection system also capable of determining if either the bioelectrical impulse sensor or the piezoelectric member is malfunctioning and, thereby determining if the input from the user has been asserted even when one of the bioelectrical impulse sensor or the piezoelectric member is malfunctioning.

14. The electromyogram interface of claim 13, in which the detection system comprises a processor that compares a current state of the first signal and the second signal to a previous state of the first signal and the second signal to determine if either of the bioelectrical impulse sensor or the piezoelectric member is malfunctioning.

15. A method of validating an electromyogram signal, comprising the steps of:
a. incrementing a counter at a first rate of a first preselected number of counts per second if the electromyogram signal has been asserted;
b. decrementing the counter at a second rate of a second preselected number of counts per second if the electromyogram signal has not been asserted and if the counter has a value not equal to zero; and
c. asserting an electromyogram state change signal if the counter has a value of not less than a predetermined threshold value, not equal to zero.

16. The method of claim 15, wherein the first rate is greater than the second rate.

17. A method of processing electromyogram information on a computer-based system that includes a computer display, comprising the steps of:
a. causing a cursor displayed on the computer display to move in response to a first assertion of an electromyogram signal;
b. displaying a sleep-mode icon on the display;
c. entering the computer-based system into a sleep-mode state when the cursor is in a position corresponding to the sleep-mode icon;
d. disabling a predetermined set of functions controlled by the computer-based system upon entering the sleep-mode state;
e. sensing a second assertion of the electromyogram signal; and
f. re-enabling the predetermined set of functions when the second assertion of the electromyogram signal indicates that a predetermined electromyogram state has been changed.

18. The method of claim 17, wherein determining if the predetermined electromyogram state has been changed is perfonned by executing a set of steps comprising:
a. incrementing a counter at a first rate of a first preselected number of counts per second if the electromyogram signal has been asserted;
b. decrementing the counter at a second rate of a second preselected number of counts per second if the electromyogram signal has not been asserted and if the counter has a value not equal to zero; and
c. asserting an electromyogram state change signal if the counter has a value of not less than a predetermined threshold value, not equal to zero.

19. The method of claim 18, wherein the first rate is greater than the second rate.

20. A method of processing electromyogram information on a computer-based system that includes a computer display, comprising the steps of:
a. causing a cursor displayed on the computer display to move in response to a first assertion of an electromyogram signal;
b. displaying a special mode icon on the display;
c. entering the computer-based system into a special mode state when the cursor is in a position corresponding to the special mode icon such that a predetermined electromyogram state change signal has been asserted; and
d. generating a special mode indication when the computer-based system has entered the special mode state.

21. The method of claim 20, further comprising determining if the predetermined electromyogram state change signal has been asserted by executing a set of steps comprising:
a. incrementing a counter at a first rate of a first preselected number of counts per second if the electromyogram signal has been asserted;
b. decrementing the counter at a second rate of a second preselected number of counts per second if the electromyogram signal has not been asserted and if the counter has a value not equal to zero; and
c. asserting an electromyogram state change signal if the counter has a value of not less than a predetermined threshold value, not equal to zero.

22. The method of claim 21, wherein the first rate is greater than the second rate.

23. The method of claim 22, wherein the special mode icon comprises an alarm mode icon and wherein the special mode state comprises an alarm state and wherein the special mode indication comprises an alarm.

24. The method of claim 22, wherein the special mode icon comprises a sleep mode icon and wherein the special mode state comprises a sleep mode state and wherein the step of generating a special mode indication comprises suppressing a predetermined set of functions until a valid termination of sleep mode is sensed.

25. A method of processing an electromyogram information from a user, comprising the steps of:
a. measuring a first electromyogram signal corresponding to a first condition from the user;
b. measuring a second electromyogram signal corresponding to a second condition from the user, the second condition contrasting with the first condition;
c. applying a fast Fourier transform to the first signal, thereby generating a first frequency domain signal;
d. applying a fast Fourier transform to the second signal, thereby generating a second frequency domain signal;
e. comparing the first frequency domain signal and the second frequency domain signal according to predefined criteria, thereby creating a filter function;
f. applying a fast Fourier transform to a real-time electromyogram signal, thereby generating a real time frequency domain signal;
g. applying the filter function to the real-time frequency domain signal, thereby generating a real-time filtered signal;
h. applying an inverse fast Fourier transform to the real-time filtered signal, thereby generating a real-time filtered time domain signal corresponding to the real-time electromyogram signal.

26. The method of claim 25, wherein the first frequency domain signal comprises a plurality of first frequency components and the second frequency domain signal comprises a plurality of second frequency components, and wherein the step of comparing the first frequency domain signal and the second frequency domain signal according to predefined criteria further comprises the steps of:
a. performing a comparison of each of the first frequency components to a corresponding one of the second frequency components to determine a frequency component difference value for each comparison:
i. setting a frequency component multiplier equal to a first multiplier value if the frequency component difference value is less than a first threshold value;
ii. setting the frequency component multiplier equal to a second multiplier value, not equal to the first multiplier value, if the frequency component difference value is not less than the first threshold value, but less than a second threshold value; and
iii. setting the frequency component multiplier equal to a third multiplier value, different from the first multiplier value and the second multiplier value, if the frequency component difference value is not less than the second threshold value; and
b. defining the filter function as multiplying each frequency component of a frequency domain signal by the frequency component multiplier corresponding to the frequency component.

27. The method of claim 26, wherein the step of applying the filter function to the real-time frequency domain signal comprises multiplying each frequency component of the real-time frequency domain signal by the frequency component multiplier corresponding to the frequency component.

28. A device for interfacing an electromyogram to a computer, comprising:
a. a power supply;
b. a first electromyogram channel input;
c. a first output, capable of transmitting a signal from the first electromyogram channel input to the computer;
d. a first computer signal input, capable of receiving a data signal from the computer;
e. a first switch output; and
f. a first relay, activated by the first computer signal input, that electrically couples the power supply to the first switch output when a first signal is asserted at the first computer signal input, the first signal indicating that a bioelectrical impulse has been sensed by the first electromyogram channel input.

29. The device of claim 28, wherein the first output is compatible with a PCMCIA card.

30. The device of claim 28, further comprising
a. a second electromyogram channel input;
b. a second output, capable of transmitting a signal from the second electromyogram channel input to the computer;
c. a second computer signal input, capable of receiving a data signal from the computer;
d. a second switch output; and
e. a second relay, activated by the second computer signal input, that electrically couples the power supply to the second switch output when a second signal is asserted at the second computer signal input, the second signal indicating that a bioelectrical impulse has been sensed by the second electromyogram channel input.

31. The device of claim 30, wherein the second output is compatible with a PCMCIA card.

32. The device of claim 30, further comprising
a. a third electromyogram channel input; and
b. a third output, capable of transmitting a signal from the third electromyogram channel input to the computer.

33. The device of claim 32, wherein the second output is compatible with a PCMCIA card.

34. An electromyogram interface for sensing an input from a user, comprising:
a. a bioelectrical impulse sensor, capable of generating a first signal when a bioelectrical impulse is asserted;
b. a piezoelectric member, capable of generating a second signal when subjected to a mechanical force corresponding to a muscle movement; and
c. a detection system, responsive to the bioelectrical impulse sensor and the piezoelectric member, that determines if the input from the user has been asserted based on the first signal and the second signal, the circuit also capable of determining if either the bioelectrical impulse sensor or the piezoelectric member is malfunctioning and, thereby determining if the input from the user has been asserted even when one of the bioelectrical impulse sensor or the piezoelectric member is malfunctioning.

35. The electromyogram interface of claim 34, in which the detection system comprises a processor that compares a current state of the first signal and the second signal to a previous state of the first signal and the signal to determine if either of the bioelectrical impulse sensor or the piezoelectric member is malfunctioning.
